(19) United States
(12) Patent Application Publication
(10) Pub. No.: US 2004/0127876 A1
(43) Pub. Date: Jul. 1, 2004

(54) Disposable absorbent garment

(75) Inventor: Robert A. Stevens, Menasha, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/335,011

(22) Filed: Dec. 31, 2002

(51) Int. Cl. A61F 13/15; A61F 13/20

(52) U.S. Cl. 604/385.3

(57) ABSTRACT

A disposable absorbent garment of this invention comprises an absorbent chassis having a front waist region, a back waist region and a crotch region extending longitudinally therebetween. A support panel extends longitudinally from one of the waist regions of the absorbent chassis and may be positioned in a folded-down condition in which the support panel overlaps the one waist region on the outside of the absorbent chassis. The support panel is elastically stretchable in a direction generally transverse to a longitudinal axis of the absorbent chassis. A fastening system is provided for securing the support panel in a chassis supporting position in which it is in its folded-down condition and elastically stretched girthwise around opposite hips of a person wearing the garment to support the absorbent chassis on the person.

### BACKGROUND OF THE INVENTION

This invention relates generally to disposable absorbent garments such as training pants, diapers, adult incontinence products and the like, and more particularly to an elastic support system for holding the absorbent structure of such garments close to the body without restricting movement of the body.

Conventionally, disposable absorbent garments have been manufactured using either a machine-direction (MD) process or a cross machine-direction (CD) process. Each of these processes has advantages and drawbacks.

In the MD process, absorbent garments are made end-to-end in continuous fashion. Elastic components of the garments which stretch end-to-end in the machine direction (e.g., leg and flap elastics) are easily applied in a stretched state to the absorbent chassis, but the elastic components that stretch in the cross-machine direction (e.g., the waist and side panel elastics) must first be turned 90 degrees and applied in a relaxed state. Typically, all of the elastic components (except for the flap components in a training pant) are on the perimeter of the garment. In this design, the waist and leg elastics and any side panel elastics provide initial fit and fit maintenance, but their impact is limited to the sides and top of the garment. This design requires highly engineered elastic systems to deliver high levels of stretch in these localized areas. When properly engineered, the result is a form-fitting garment with a generally smooth external appearance.

In the CD process, the garments are made side-by-side in continuous fashion. Typically in this process all elastic components, except the flap elastics in a training pant, can be applied in a stretched state, in the machine direction, and across the absorbent structure, thus making elastic application and control relatively easy. Furthermore, since the elastics can be stretched completely across the absorbent, high levels of local stretch are not needed and elasticity requirements are reduced. However, this design attaches the form fitting elastics to the outer cover of the absorbent structure, causing the absorbent to bunch up during elastic retraction. This, in turn, results in a relatively loose, blousy appearance which is not as acceptable as the more form fitting appearance of the MD process.

An alternative approach to the CD process has been to apply form fitting elastics as an extra elastic band of material above the absorbent structure, resulting in a design where the absorbent structure is slung below the elastic “belly band”. This arrangement, however, has limited absorbent functionality due to the relatively small absorbent size and positioning outside the male target zone.

There is a need, therefore, for an improved disposable absorbent garment design which overcomes the problems noted above.

### SUMMARY OF THE INVENTION

In general, a disposable absorbent garment of this invention comprises an absorbent chassis having a front waist region, a back waist region and a crotch region extending longitudinally therebetween. A support panel extends longitudinally from one of the waist regions of the absorbent chassis and may be positioned in a folded-down condition in which the support panel overlaps the one waist region on the outside of the absorbent chassis. The support panel is elastically stretchable in a direction generally transverse to a longitudinal axis of the absorbent chassis. A fastening system is provided for securing the support panel in a chassis supporting position in which it is in its folded-down condition and elastically stretched girthwise around opposite hips of a person wearing the garment to support the absorbent chassis on the person.

In a particular embodiment, the absorbent chassis of the disposable absorbent garment described above has lateral sides which define, at least partially, leg openings when the garment is worn, and leg elastic members along the lateral sides of the chassis adjacent the leg openings. The support panel and the leg elastic members combine to substantially completely surround the leg openings when the garment is worn.

In one embodiment, the garment of the present invention is a training pant. In other embodiments, the garment may be a diaper or an incontinence product (e.g., an adult care product).

Other features of this invention will be in part apparent and in part pointed out hereinafter.

### DEFINITIONS

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Connected” refers to the joining, adhering, bonding, attaching, or the like, of two elements. Two elements will be considered to be connected together when they are connected directly to one another or indirectly to one another, such as when each is directly connected to intermediate elements.

“Cross-machine direction process” refers to an assembly process in which garments travel through the process in a side-by-side orientation.

“Disposable” refers to articles which are designed to be discarded after a limited use rather than being laundered or otherwise restored for reuse.

“Disposed,” “disposed on,” and variations thereof are intended to mean that one element can be integral with another element, or that one element can be a separate structure bonded to or placed with or placed near another element.

“Elastic,” “elasticized” and “elasticity” mean that property of a material or composite by virtue of which it tends to recover its original size and shape after removal of a force causing a deformation.

“Elastomeric” refers to a material or composite which can be elongated by at least 25 percent of its relaxed length and which will recover, upon release of the applied force, at least 10 percent of its elongation. It is generally preferred that the elastomeric material or composite be capable of being elongated by at least 100 percent, more preferably by at least 300 percent, of its relaxed length and recover, upon release of an applied force, at least 50 percent of its elongation.

“Fabrics” is used to refer to all of the woven, knitted and nonwoven fibrous webs.

“Flexible” refers to materials which are compliant and which will readily conform to the general shape and contours of the wearer's body.

“Force” includes a physical influence exerted by one body on another which produces acceleration of bodies that are free to move and deformation of bodies that are not free to move. Force is expressed in grams per unit area.

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90° are designated “wettable” or hydrophilic, while fibers having contact angles greater than 90° are designated “nonwettable” or hydrophobic.

“Integral” is used to refer to various portions of a single unitary element rather than separate structures bonded to or placed with or placed near one another.

“Inward” and “outward” refer to positions relative to the center of an absorbent article, and particularly transversely and/or longitudinally closer to or away from the longitudinal and transverse center of the absorbent article.

“Layer” when used in the singular can have the dual meaning of a single element or a plurality of elements.

“Liquid impermeable”, when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable” when used herein.

“Longitudinal” and “transverse” have their customary meaning. The longitudinal axis lies in the plane of the garment and is generally parallel to a vertical plane that bisects a standing wearer into left and right body halves when the article is worn. The transverse axis lies in the plane of the article generally perpendicular to the longitudinal axis. The garment as illustrated is longer in the longitudinal direction than in the transverse direction.

“Machine direction process” refers to an assembly process in which garments travel through the process in an end-to-end or waist-to-waist orientation.

“Member” when used in the singular can have the dual meaning of a single element or a plurality of elements.

“Nonwoven” and “nonwoven web” refer to materials and webs of material which are formed without the aid of a textile weaving or knitting process.

“Operatively joined,” with reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Permanently bonded” refers to the joining, adhering, connecting, attaching, or the like, of two elements of an absorbent garment such that the elements tend to be and remain bonded during normal use conditions of the absorbent garment.

“Refastenable” refers to the property of two elements being capable of releasable attachment, separation, and subsequent releasable reattachment without substantial permanent deformation or rupture.

“Releasably attached,” “releasably engaged” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Rupture” means the breaking or tearing apart of a material; in tensile testing, the term refers to the total separation of a material into two parts either all at once or in stages, or the development of a hole in some materials.

“Stretch bonded” refers to an elastic member being bonded to another member while the elastic member is extended at least about 25 percent of its relaxed length. Desirably, the term “stretch bonded” refers to the situation wherein the elastic member is extended at least about 100 percent, and more desirably at least about 300 percent, of its relaxed length when it is bonded to the other member.

“Stretch bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered.

“Surface” includes any layer, film, woven, nonwoven, laminate, composite, or the like, whether pervious or impervious to air, gas, and/or liquids.

“Tension” includes a uniaxial force tending to cause the extension of a body or the balancing force within that body resisting the extension.

These terms may be defined with additional language in the remaining portions of the specification.

### BRIEF DESCRIPTION OF THE DRAWINGS

FIG. 1 is a perspective of one embodiment of a child's training pant of this invention with a support panel folded down on the back waist region of the pant and secured in a chassis supporting position in which the support panel is stretched around the hips of the wearer to support the absorbent chassis of the pant close to the body;

FIG. 2 is a top plan view of the training pant in its unfastened, relaxed and laid flat condition to show the surface of the training pant which faces the wearer when the training pant is worn, with interior components of the training pant being shown in hidden lines;

FIG. 3 is a bottom plan view of the training pant in its unfastened, relaxed and laid flat condition to show the surface of the training pant which faces away from the wearer when the training pant is worn;

FIG. 3A is a sectional view taken in the plane of lines 3A--3A of FIG. 3, the spacing between the laminated components being exaggerated to show details;

FIG. 4 is a view similar to FIG. 3 but showing the support panel in its folded-down condition;

FIG. 5 is a front elevation of the training pant with the support panel in a folded-down and stretched condition in which one end of the panel is fastened to the front waist region and the other end of the panel is unfastened, and without gaps between the lateral side edges of the front and back waist regions;

FIG. 6 is a view of a portion of FIG. 2 showing the support panel in a stretched condition;

FIGS. 7-9 are front elevations of the training pant showing the support panel secured in various supporting positions using cooperating fastening components;

FIGS. 10-14 are front elevations of training pants illustrating different fastening component arrangements for securing the support panel in its supporting position;

FIG. 15 is a front elevation of a training pant showing the support panel folded down on a fold line spaced above the back waist region of the pant;

FIG. 16 is a front elevation of a training pant showing the support panel in a supporting position in which a lower portion of the panel is folded up under an upper portion of the panel to create a double-thick support panel for providing increased pressure at the waist area;

FIG. 17 is a view similar to FIG. 16 showing a different embodiment of a double-thick support panel; and

FIG. 18 is a front elevation of a training pant in which the support panel in its supporting position has overlapping end sections.

Corresponding parts are designated by corresponding numbers throughout the drawings.

### DETAILED DESCRIPTION OF THE DRAWINGS

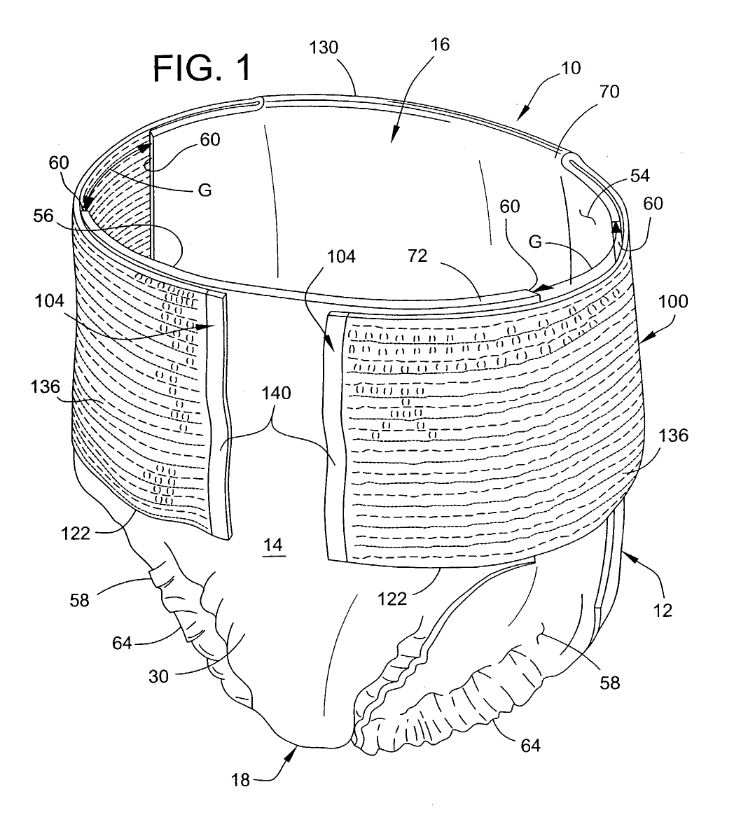

The features of the present invention can be incorporated into a variety of disposable absorbent garments but are especially applicable to training pants, diapers and adult care products. For ease of explanation, the present invention is hereafter described in connection with a child's training pant, generally indicated by the reference number 10 in FIG. 1. In particular, the methods and apparatus will be described in terms of those for making disposable training pants, such as those constructed using the methods and apparatus disclosed in U.S. Pat. No. 4,940,464 issued Jul. 10, 1990 to Van Gompel et al.; U.S. Pat. No. 5,766,389 issued Jun. 16, 1998 to Brandon et al.; and as described in U.S. patent application Ser. No. 09/954,444 by Swanton et al. titled “Method and Apparatus for Assembling Refastenable Absorbent Garments”, filed Sep. 14, 2001; and U.S. patent application Ser. No. 09/954,478 by Sanders titled “Method and Apparatus for Assembling Refastenable Absorbent Garments”, filed Sep. 14, 2001; all of which disclosures are incorporated herein by reference for all purposes not inconsistent with the present invention.

With reference now to the drawings, and in particular to FIGS. 1-3, a training pant 10 comprises an absorbent chassis, generally designated 12, having a lengthwise, longitudinal direction Y, a lateral, transverse cross-direction X, a front waist region 14, a back waist region 16, and an intermediate crotch region 18 interconnecting the front and back waist regions. The waist regions 14 and 16 comprise those portions of the pant 10 which when worn, wholly or partially cover or encircle the waist or mid-lower torso of the wearer. The intermediate crotch region 18 lies between and interconnects the waist regions 14 and 16, and comprises that portion of the article 10 which, when worn, is positioned between the legs of the wearer and covers the lower torso of the wearer. Thus, the intermediate crotch region 18 and the lower area of front region 14 are areas where repeated fluid surges typically occur in the training pant or other disposable absorbent article when the wearer is in an upright position.

The pant 10 includes a substantially liquid-impermeable outer cover member 30, a liquid-permeable bodyside liner 28, and an absorbent body structure, generally designated 32, sandwiched between the outer cover member 30 and the bodyside liner layer 28. The absorbent body structure may be secured to the outer cover member 30 by an adhesive and secured to the bodyside liner 28 by an adhesive. In certain embodiments, a surge layer 48 may be optionally located adjacent the absorbent body structure and attached by way of adhesive.

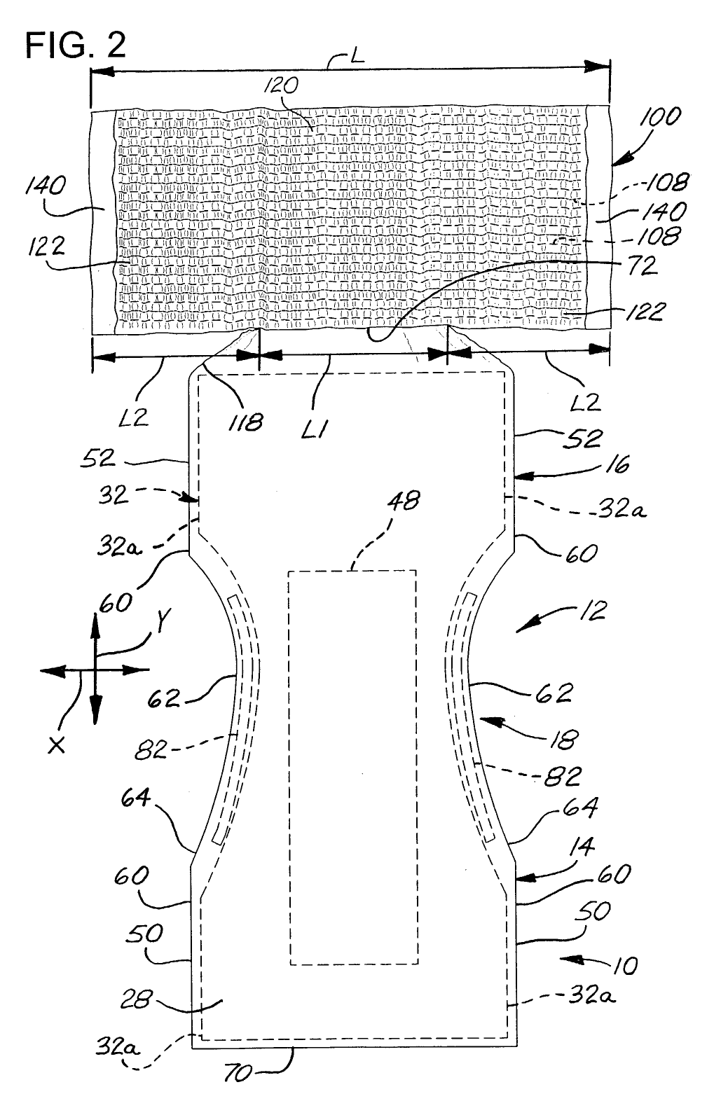

In the illustrated embodiment of the pant 10, as shown in FIG. 2, the front and back waist regions 14, 16 of the chassis 12 include front ear portions 50 extending laterally outward at opposite sides of the front waist region and back ear portions 52 extending laterally outward at opposite sides of the back waist region. In the illustrated embodiment, the ear portions are an extension of the chassis structure and may be, for example, be extensions of the outer cover member 30, bodyside liner 28, or both. The absorbent body structure 32 includes laterally extending wing portions 32a, as seen in hidden lines in FIG. 2. The wing portions extend at least partially into the ear portions 50, 52.

With the training pant 10 fastened on a wearer, as illustrated in FIG. 1, the absorbent chassis 12 defines a three-dimensional pant configuration having an interior space 54 for receiving the lower torso of a person wearing the pant, a waist opening 56 for receiving the wearer into the interior space of the pant, and a pair of leg openings 58. In one embodiment, the lateral sides 60 of the waist regions 14, 16 cooperate with the lateral sides 62 of the crotch region 18 of the chassis 12 to define curved leg contours 64 (see FIG. 2). The curved leg contours 64 define the leg openings 58 when the article is assembled. In some configurations the lateral sides 60 are unconnected. In other configurations the lateral sides 60 may be bonded in a known manner so as to define side seams (not shown) of the pant structure. With this type of configuration, the pant 10 is pulled on by the wearer in a manner similar to underwear. Desirably, these side seams may be separable or tearable so that the pant 10 may be removed from the wearer by tearing the seams and removing the article in a manner similar to a diaper. In still other embodiments, the front and back ear portions 50, 52 may be separable and re-attachable at the side seams.

FIG. 2 shows a plan view of the representative training pant 10 in its generally flat-out, relaxed state. In this view, the bodyside surface of the pant 10 which contacts the wearer is facing the viewer. The outer edges of the chassis 12 define a periphery with lateral sides extending generally in the Y direction and longitudinal ends 70, 72 extending generally in the X direction. The longitudinal ends 70, 72 are shown as straight, but optionally, may be curvilinear. The lateral sides are defined by the lateral sides 60 of the front and back ear portions 50, 52, and lateral sides 62 of the crotch region 18. Elastomeric gathering members, such as leg elastics 82 may be provided, as is well known in the art. (For convenience of illustration, the gathers induced by the gathering members 82 are not shown in FIG. 2.) The liner 28, outer cover 30, absorbent structure 32, surge layer 48, and elastic members 82 may be assembled together into a variety of well-known absorbent article configurations.

The leg elastic members 82 may be located in the lateral side margins of the chassis 12, particularly along the lateral sides 62 of the crotch region 18, and are configured to draw and hold the chassis 12 against the legs of the wearer. The elastic members 82 are secured to the chassis 12, for example with an adhesive, in an elastically extended state so that in a normal under-strain condition, the elastic members 82 effectively contract against the chassis. The use of leg elastic members in absorbent articles such as disposable diapers and training pants is widely known and understood in the art.

In the particular embodiments shown in the drawings, the training pant 10 does not include conventional elastic waistbands or waist elastics. As will be apparent from the description which follows, the use of such elastics is unnecessary, although they may be used if desired.

The liner 28 and outer cover member 30 may be generally coextensive, and may have length and width dimensions which are generally larger than and extend beyond the corresponding dimensions of the absorbent structure 32. The extending portions define the corresponding side and end margins that allow for the leg elastics 82 and side seams (if used) to attach together the outer cover member 30 and liner 28. Optionally, the bodyside liner 28 and outer cover member 30 may not be coextensive. The outer cover member 30 may be composed of a liquid permeable material, but desirably comprises a material which is configured to be substantially impermeable to liquids. For example, a typical outer cover can be manufactured from a thin plastic film, a composite laminate, or other flexible, substantially liquid-impermeable material.

Alternative constructions of the outer cover member 30 may comprise a woven or non-woven fibrous web layer which has been totally or partially constructed or treated to impart the desired levels of liquid impermeability to selected regions that are adjacent or proximate the absorbent body. For example, the outer cover may include a gas-permeable, nonwoven fabric layer laminated to a polymer film layer which may or may not be gas-permeable. Other examples of fibrous, cloth-like outer cover materials can comprise a stretch thinned or stretch thermal laminate material. Although the outer cover 30 typically provides the outermost layer of the article, optionally the article may include a separate outer cover component member which is additional to the outer cover member.

The outer cover may be of a non-stretch or stretch material. In a particular embodiment, the outer cover 30 may be formed substantially from an elastomeric material and is thus stretchable. In this embodiment, the outer cover 30 may define the entire front and back waist regions 14, 16, including the front and back ear portions 50, 52. The outer cover 30 may, for example, be composed of a single layer, multiple layers, laminates, spunbond fabrics, films, meltblown fabrics, elastic netting, microporous web, bonded carded webs or foams comprised of elastomeric or polymeric materials. Elastomeric nonwoven laminate webs may include a nonwoven material joined to one or more gatherable nonwoven webs, films, or foams. Stretch Bonded Laminates (SBL) and Neck Bonded Laminates (NBL) are examples of elastomeric composites. Nonwoven fabrics are any webs of material which are formed without the use of a textile weaving or knitting process and which instead comprise individual fibers and threads which are interlaid but not in an identifiable repeating manner. Examples of suitable materials are Spunbond-Meltblown fabrics, Spunbond-Meltblown-Spunbond fabrics, Spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The elastomeric materials may include PEBAX® elastomer (available from AtoChem located in Philadelphia, Pa.), HYTREL® elastomeric polyester (available from E. I. DuPont de Nemours located in Wilmington, Del.), KRATON® elastomer (available from Shell Chemical Company located in Houston, Tex.), or strands of LYCRA® elastomer (available from E. I. DuPont de Nemours located in Wilmington, Del.), or the like, as well as combinations thereof. The outer cover 30 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

The bodyside liner 28 presents a body-facing surface which is compliant, soft-feeling, and non-irritating to the wearer's skin. Further, the bodyside liner 28 can be less hydrophilic than the absorbent body 32, and is sufficiently porous to be liquid permeable, permitting liquid to readily penetrate through its thickness to reach the absorbent body 32. A suitable bodyside liner layer 28 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner layer 28 is typically employed to help isolate the wearer's skin from liquids held in absorbent body 32.

Various woven and nonwoven fabrics can be used for bodyside liner 28. For example, the bodyside liner may include a meltblown web, a spunbonded web, or a bonded-carded-web composed of the desired fibers. The various fabrics can be composed of natural fibers, synthetic fibers or combinations thereof. In particular aspects, the bodyside liner 28 may be comprised of polymer fibers, networks, laminates, liquid permeable films, cellulosic fibers, rayon, water swellable gels, and elastomeric materials, as well as combinations thereof. Suitable materials for the bodyside liner can include meltblown webs, airlaid webs, spunbond webs, or bonded-carded webs of synthetic continuous or discrete polymer fibers and/or natural fibers, a pattern bonded spunbonded web, airlaid web, or bonded carded web, as well as combinations thereof. Suitable polymers can include polypropylene, polyethylene, polyester, and bicomponent materials composed of these polyolefins.

The bodyside liner fabrics may be composed of a substantially hydrophobic material, and the hydrophobic material may optionally be treated with a surfactant or otherwise processed to impart a desired level of wettability and hydrophilicity. In a particular embodiment of the invention, the bodyside liner 28 can be a nonwoven, spunbond polypropylene fabric which is necked approximately 60%. Strands of KRATON® G2760 elastomer material may be adhered to the necked spunbond material. The fabric can be surface treated with an operative amount of surfactant, such as about 0.45% AHCOVEL® Base N62 surfactant, available from Uniqema, a division of ICI, a business having offices located in New Castle, Del. The surfactant can be applied by any conventional means, such as spraying, printing, brush coating or the like.

In particular embodiments wherein it is desired that the bodyside liner layer 28 be stretchable, suitable elastomeric materials can include elastic strands, LYCRA® elastics, elastic films, cast or blown; nonwoven elastic webs, meltblown or spunbond elastomeric fibrous webs, as well as combinations thereof. Examples of elastomeric materials include KRATON® elastomers, HYTREL® elastomers, ESTANE® elastomeric polyurethanes (available from B.F. Goodrich and Company located in Cleveland, Ohio), or PEBAX® elastomers. The bodyside liner may include blends or laminates of fibers, scrim, webs, and films with perforations, apertures, creping, heat activation, embossing, micro-straining, chemical treatment, or the like, as well as combinations thereof.

The bodyside liner 28 and outer cover 30 are connected or otherwise associated together in an operable manner. As used herein, the term “associated” encompasses configurations in which the bodyside liner 28 is directly joined to the outer cover 30 by affixing the bodyside liner 28 directly to the outer cover 30, and configurations wherein the bodyside liner 28 is indirectly joined to the outer cover 30 by affixing the bodyside liner 28 to intermediate members which in turn are affixed to the outer cover 30. The bodyside liner 28 and outer cover 30 can, for example, be operatively joined to each other in at least a portion of the training pant periphery by suitable attachment mechanisms (not shown) such as adhesive bonds, sonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the bodyside liner 28 to the outer cover 30. It should be readily appreciated that the above-described attachment means may also be employed to suitably interconnect, assemble and/or affix together the various other component parts of the articles which are described herein.

The absorbent body structure 32 (including wing portions 32a and optional surge layer 48) is positioned between the outer cover 30 and the bodyside liner 28. The absorbent body structure 32 can be any structure or combination of components which are generally compressible, conformable, non-irritating to a wearer's skin, and capable of absorbing and retaining liquids and certain body wastes. For example, the structure 32 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from U.S. Alliance of Childersburg, Ala., USA, and is a bleached, highly absorbent sulfate wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art.

As a general rule, the superabsorbent material is present in the absorbent web in an amount of from about 0 to about 90 weight percent based on total weight of the web. In particular, the web may have a density within the range of about 0.10 to about 0.35 grams per cubic centimeter. Alternately, the density of the web may be even higher.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and desirably is capable of absorbing more than about 25 times its weight in liquid. Suitable superabsorbent materials are readily available from various suppliers. For example, Favor 880 superabsorbent is available from Stockhausen GmbH of Germany; and Drytech 2035 is available from Dow Chemical Company of Midland, Mich., USA.

After being formed or cut into a desired shape, the absorbent web material may be wrapped or encompassed by a suitable tissue wrap that aids in maintaining the integrity and shape of the absorbent structure 32.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. Nos. 4,100,324 to Anderson, et al.; 5,284,703 to Everhart, et al.; and 5,350,624 to Georger, et al.; which are incorporated herein in their entirety by reference thereto for all purposes.

As mentioned, the absorbent body structure 32 may also include a surge management layer 48 which helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent structure. The surge layer can be located below the bodyside liner layer 28. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference in their entirety for all purposes.

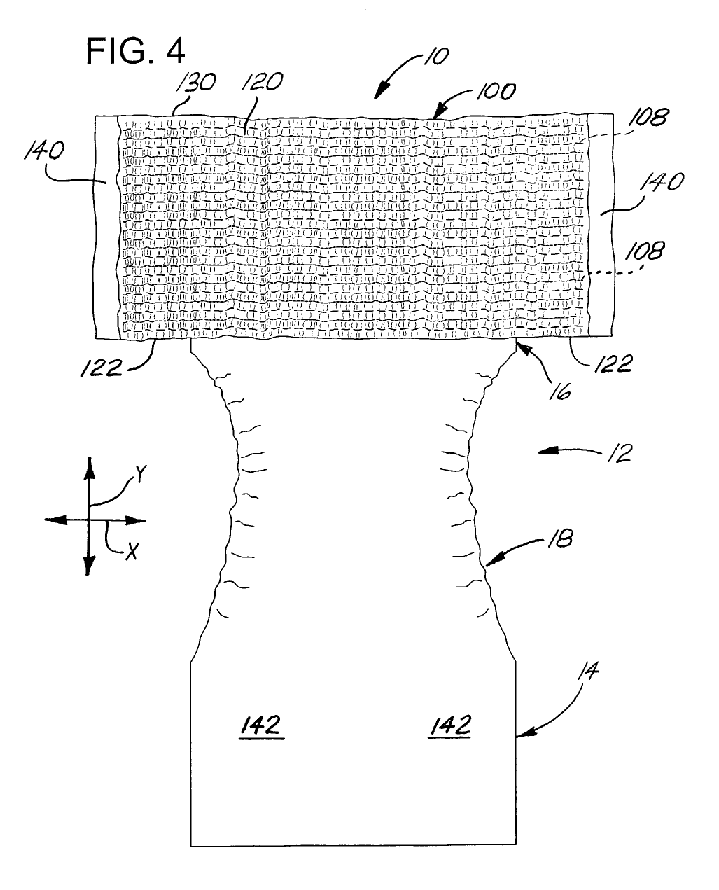

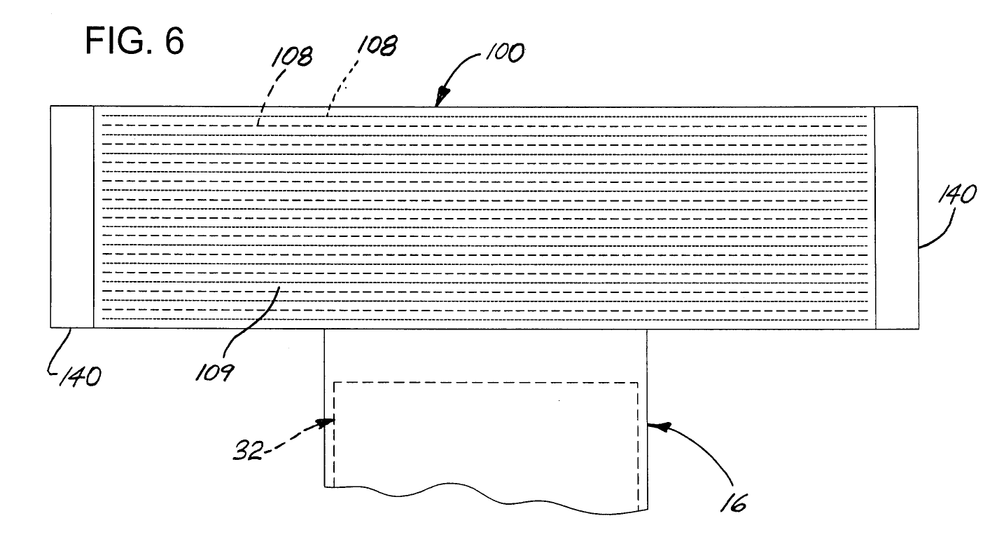

In accordance with one aspect of the present invention, the training pant 10 includes a support panel, generally indicated at 100, of elastically stretchable material extending longitudinally from one or both of the waist regions 14, 16 of the chassis 12. In the particular embodiment shown in FIGS. 1-6, the support panel 100 extends only from the back waist region 16, but it will be understood that the support panel could extend from the front waist region 14 only, or that a support panel could extend from each of the front and back waist regions. In the particular configuration shown in FIGS. 1-6, involving only a back support panel 100, the support panel is adapted to be positioned in a folded-down condition in which the panel overlaps the back waist region 16 on the outside of the absorbent chassis 12 (FIG. 1), and then elastically stretched around the hips of the wearer on the outside of the absorbent chassis for attachment to the front waist region 14 in a chassis supporting position by means of a fastening system generally designated 104. When secured in this position, the support panel 100 provides full elastic support to the chassis 32 across the buttocks and around the hips of the wearer to effect and maintain a gentle, body-conforming fit and appearance of the training pant 10.

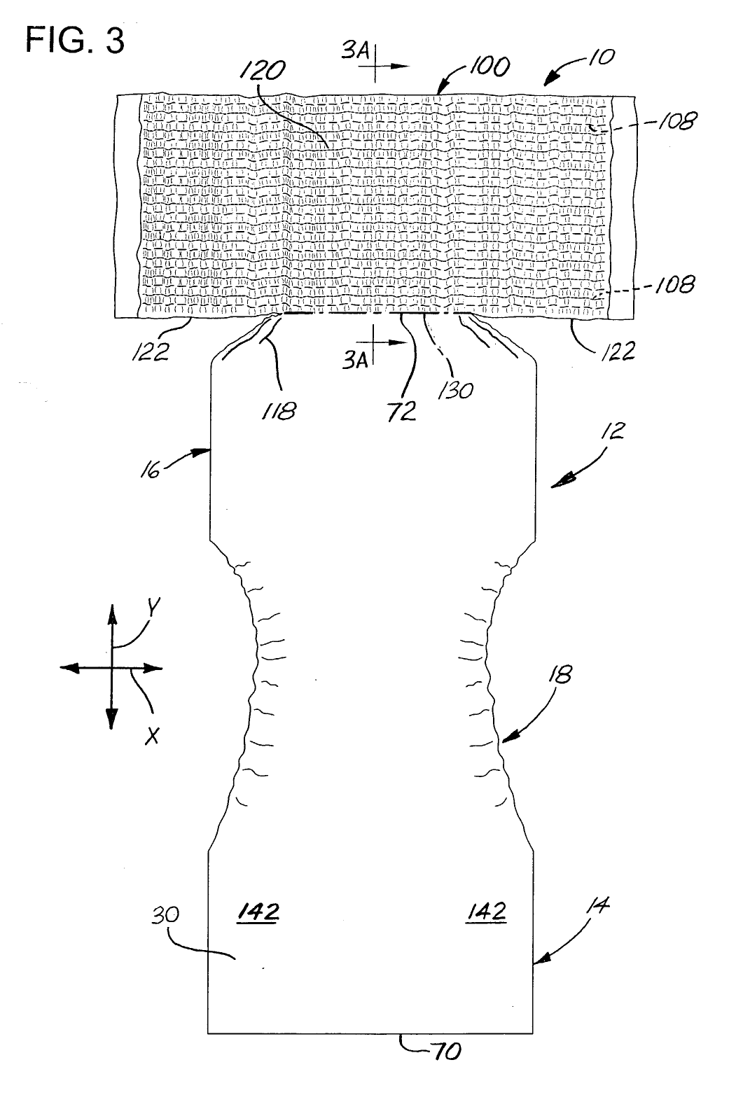

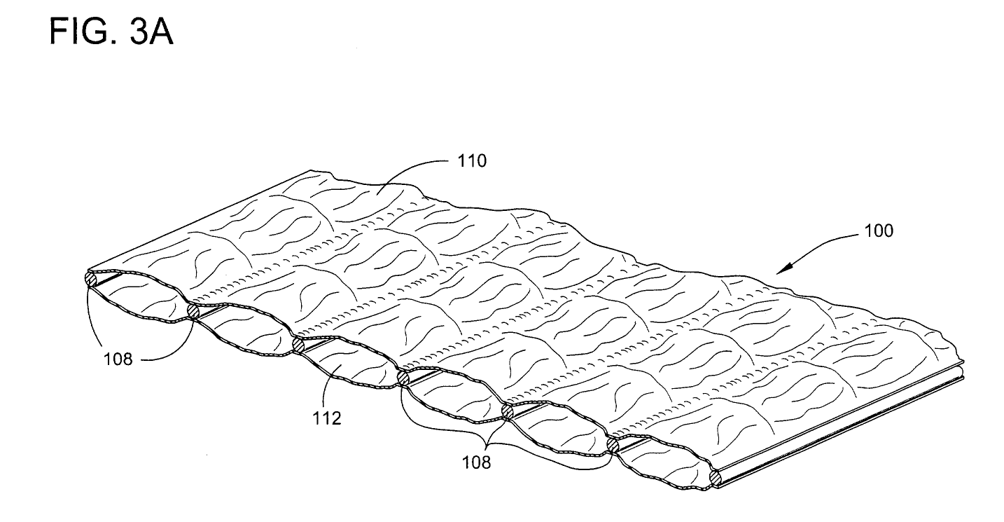

In the embodiment shown in FIGS. 2 and 3, the support panel 100 is generally rectangular in shape and is attached to the back waist region 16 of the chassis 12 in a suitable manner. In one particular embodiment, the support panel 100 comprises elastic elements 108, e.g., elongate strands of LYCRA® elastomer (available from E. I. DuPont de Nemours located in Wilmington, Del.), sandwiched between first and second layers 110, 112 of a non-woven facing material (see FIG. 3A) . These layers 110, 112 may be integral extensions of the outer cover 30 and/or integral extensions of the inner bodyside liner 28. Alternatively, the layers 110, 112 may be formed separate from the chassis 12 and then permanently secured to the waist region 16 adjacent its waist edge along a seam, using attachment means known to those skilled in the art such as adhesive, thermal or ultrasonic bonding. In a particular embodiment, the elastic strands 108 extend across the support panel 100 substantially in the X direction substantially the full length of the panel and are applied in a stretched condition to the layers 110, 112 of facing material so that, when the strands assume a relaxed state, gathers 118 are formed in the layers of the support panel and portions of the back waist region adjacent waist end 72 (see FIGS. 2 and 3). As a result of these gathers, when the absorbent chassis 12 is relaxed the longitudinal chassis end 72 in the Y direction is narrower than the longitudinal chassis end 70.

In one embodiment, the support panel 100 has a center section 120 generally coextensive with the longitudinal end 72 of the waist region 16 of the chassis 12 in the X direction, and opposite end sections 122 extending laterally from the center section in the X direction. The overall length L of the support panel 100 in the X direction when the panel is in a relaxed condition (FIG. 2), including the length L1 of the center section of the panel and the lengths L2 of the two end sections 122, will vary depending on such factors as the range of waist sizes the garment is intended to fit, the stretchability of the support panel, and the tension to be exerted on the wearer by the support panel when it is stretched and secured in place. In some embodiments, the overall length L of the support panel 100 may be the same or even less than the dimension of the longitudinal end 72 of the waist region 16 in the X direction, in which case the length L2 of the end sections 122 of the support panel is zero.

In the embodiment shown in FIG. 1, when the support panel 100 is secured in place using the fastening system 104, the lateral sides 60 of the front and back waist regions 14, 16 are separated by gaps G at opposite sides of the chassis, and the gaps are bridged by respective end sections 122 of the support panel 100. The size of these gaps G may vary depending on such factors as waist size and the width of the front and back waist regions 14, 16 in the Y direction. Alternatively, the lateral sides 60 of the front and back waist regions 14, 16 may abut or overlap to form side edge seams (not shown). Where gaps G exist, the leg openings 58 are defined by the lower edges of the bridging portions of the support panel 100 and the curved leg contours 64 of the absorbent chassis 12, and the waist opening 56 is defined by the upper edges of the bridging portions of the support panel and the longitudinal ends 70, 72 of the chassis 12. In embodiments where the front and back waist regions 14, 16 overlap one another along side seams and are held in this position by the support panel 100, the leg openings 58 are defined by the curved leg contours 64 of the absorbent chassis 12, and the waist opening 56 is defined by the longitudinal ends 70, 72 of the chassis 12.

In general, the length L of the support panel should be sufficient to encompass at least two-thirds and up to 100% of the total girth of the wearer when the support panel is stretched and secured in its supporting position. Further, the support panel 100 is preferably elastically stretchable no more than 300% of its overall length L, more preferably no more than 250% of its overall length L, and even more preferably no more than 220% of its overall length L. Exemplary support panel dimensions L, L1, L2 and stretch ranges for various size training pants, diapers and adult pants are set forth in Table 1 below.

TABLE 1

| | Fit | | | | Support panel | |
|---|---|---|---|---|---|---|
| | range | L1 | L | L2 | stretch range | |
| Example | (in) | (in) | (in) | (in) | (in) | (%) |
| 1. Training | 15.6-25.2 | 6 | 6 | 0 | 9.6-19.2 | 60-220 |
| pant (size 3) | | | | | | |
| 2. Training | 15.6-25.2 | 6 | 8 | 1 | 9.6-19.2 | 20-140 |
| pant (size 3) | | | | | | |
| 3. Training | 15.6-25.2 | 8 | 8 | 0 | 0⁺-17.2 | 0⁺-115 |
| pant/diaper | | | | | | |
| 4. Training | 15.6-25.2 | 8 | 10 | 1 | 0⁺-17.2 | 0⁺-72 |
| pant/diaper | | | | | | |
| 5. Adult pant | 22-38 | 8 | 10 | 1 | 16.0-32.0 | 60-220 |
| 6. Adult pant | 22-38 | 8 | 12 | 2 | 16.0-32.0 | 33-167 |

In the above Table 1, the dimensions L, L1 and L2 correspond to the dimensions shown in FIG. 2, where the absorbent chassis 12 and support panel 100 are shown in a relaxed, unstretched condition. As shown in FIG. 2, L1 represents the X-direction dimension of the longitudinal end 72 of the chassis 12 in a relaxed condition; L represents the overall X-direction dimension of the support panel 100 in a relaxed condition; and L2 represents the X-direction dimension of each end section 122 (if any) of the support panel in its relaxed condition. The term “Support Panel Stretch Range” as used in Table 1 represents a range of stretch (in inches) of the support panel 100 over and above the dimension L when the support panel is fastened in place.

In particular, Example 1 relates to a training pant dimensioned to fit a person having a waist size ranging from 15.6 in. to 25.2 in. When this pant is worn, the sides of the front and back waist regions 14, 16 are typically spaced from one another to form gaps G at the sides of the pant, as shown in FIG. 1. In this particular example, dimensions L1, L and L2 are 6 in., 6 in. and 0 in., respectively (meaning that the relaxed support panel 100 has an overall dimension L which is equal to the X-direction dimension L1 of the longitudinal end 72 of the waist region 16). Further, the stretch range in this example is 9.6 in. to 19.2 in., meaning that the overall L dimension of the support panel 100, when in its folded-down, stretched and fastened condition, increases an amount in the range of 19.6 in. to 25.2 in., which represents a 60-220% increase in the overall length of the support panel in the X-direction. Example 2 provides similar information, but for a support panel having different L and L2 dimensions. Examples 3 and 4 relate to training pants and/or diapers where the sides of the front and back waist regions 14, 16 may or may not overlap, depending on the size of the wearer. In instances where the sides of the waist regions 14, 16 do overlap (i.e., there are no gaps G), the amount of support panel stretch may range from 0⁺, representing a nominal amount of stretch sufficient only to provide a tension necessary to hold the pant in place, to a larger amount, depending on preference. Examples 5 and 6 relate to adult incontinence pants. It will be understood that these examples 1-6 are illustrative only.

In some configurations, the support panel 100 may be elastically stretchable in one or more directions other than the X direction. For example, the panel may be stretchable in the Y direction, or at one or more angles oblique to the X and Y directions to allow the tension exerted by the support panel in these directions to be varied to take into account body shape or other factors. Further, the support panel 100 can be designed to have areas which possess different stretch characteristics, some areas being stretchable to a greater extent than other areas, for example, and/or some areas being more or less resistant to stretch than other areas, thus allowing the tension forces exerted by the panel when in its supporting position to be varied according to the shape, size and needs of the wearer. The stretch characteristics of the support panel 100 can be varied in any manner known in the art, such as by varying the type, size, orientation and/or elastic characteristics of the elastic elements or material.

The support panel 100 can have configurations other than those described above. For example, the panel can be fabricated as a single sheet of stretchable elastic material, or as multiple pieces attached to one another, some of which may be elastically stretchable and others non-stretchable. In particular embodiments, the elastic material comprises a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the names of Taylor et al.; and PCT Publication WO 01/88245 published on Nov. 22, 2001 in the names of Welch et al.; all of which are incorporated herein by reference for all purposes in a manner not inconsistent herewith. In another embodiment, the center section 120 of the support panel 100 is of a non-stretchable, inelastic material and the end sections 122 are of an elastic material suitably permanently attached to opposite sides of the center section, as by attachment means known to those skilled in the art such as adhesive, thermal or ultrasonic bonding.

The support panel 100 can be attached to the chassis 12 in any number of different ways, as by attachment means known to those skilled in the art such as adhesive, thermal or ultrasonic bonding. Certain areas, e.g., the center section 120, of the support panel may be permanently attached to the waist region 16, while other areas may be attached by n1001 on-permanent, e.g., frangible, connections to permit separation from the waist region as the panel 100 is stretched to its supporting position.

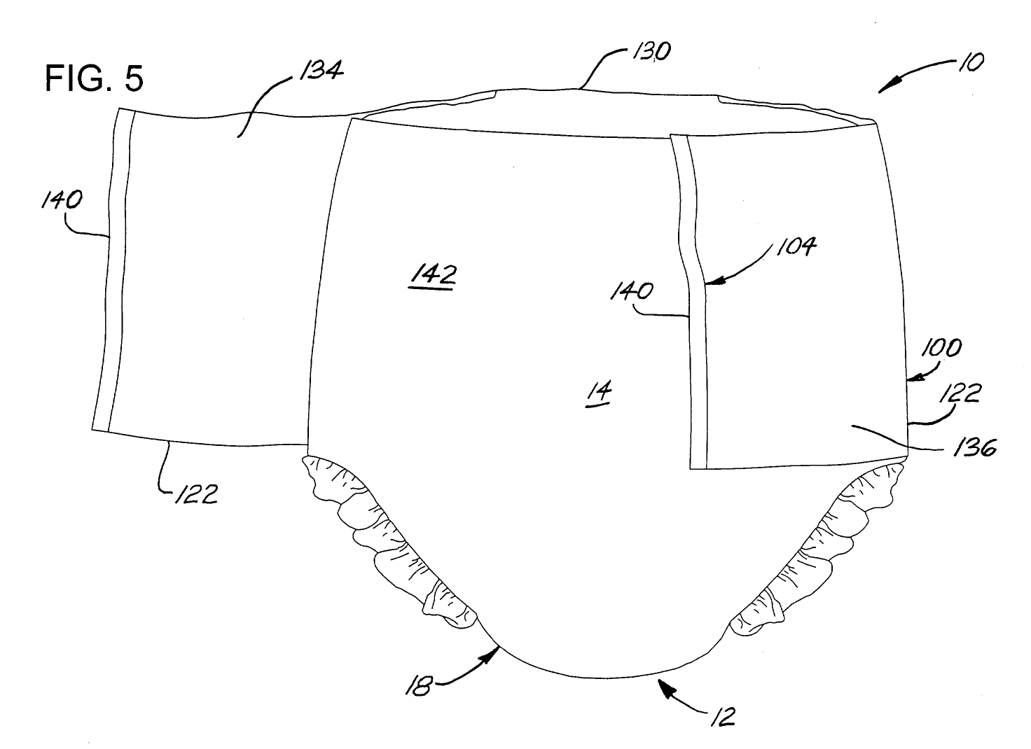

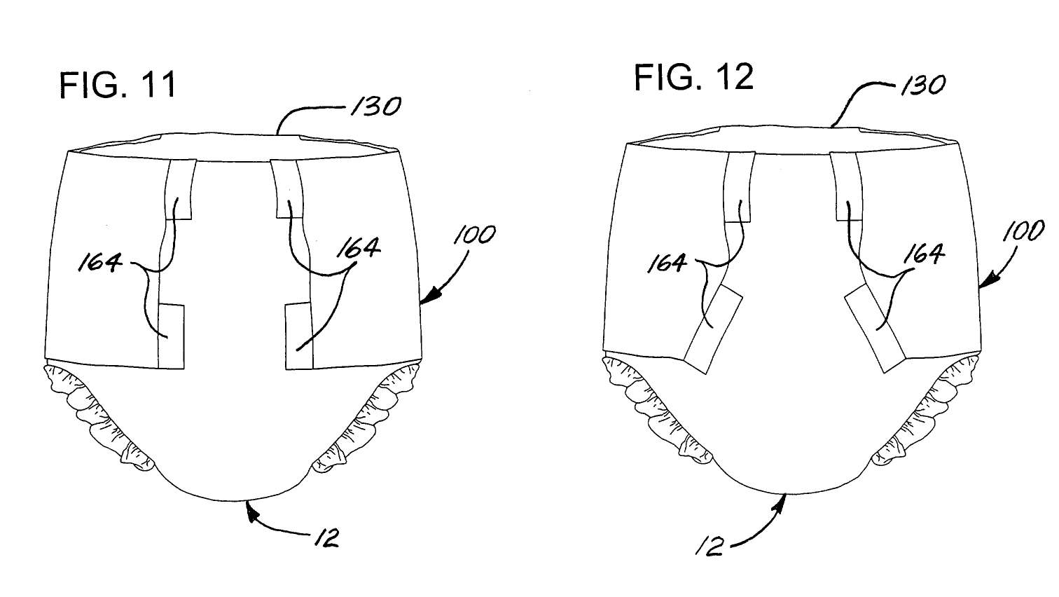

As noted above, the support panel 100 is designed to be folded down on the outside of the waist region 16 of the chassis 12, the fold preferably being along a fold line 130 extending transversely of the garment in the X direction. In particular, this fold line 130 may be generally coincident with the respective longitudinal end 70 or 72 of the waist region 16 (depending on whether the support panel extends from the front or back waist region 14, 16), or generally parallel to the longitudinal end at a location spaced from the end a suitable distance in the Y direction (e.g., see FIG. 15). When in its folded-down condition, the support panel 100 has an inner surface 134 facing toward the chassis 12 and an outer surface 136 facing away from the chassis (FIG. 5). In one embodiment (FIG. 1), the support panel 100 is sized so that when it is folded down along the fold line 130 and fastened to the front waist region, the support panel extends down substantially the full length of each of the front and back waist regions to provide full body support around the absorbent chassis 12. Further, in this embodiment the folded-down support panel 100 has a width in the Y direction sufficient to extend down to locations generally corresponding or adjacent to the leg elastic members 82 of the pant, so that the elastic support panel and leg elastic members combine to substantially completely surround the leg openings 58 to hold the pant close to the legs and thereby avoid leakage areas. In other embodiments, the support panel 100 may suitably have a width in the Y direction substantially greater or substantially less than the width shown in FIG. 1.

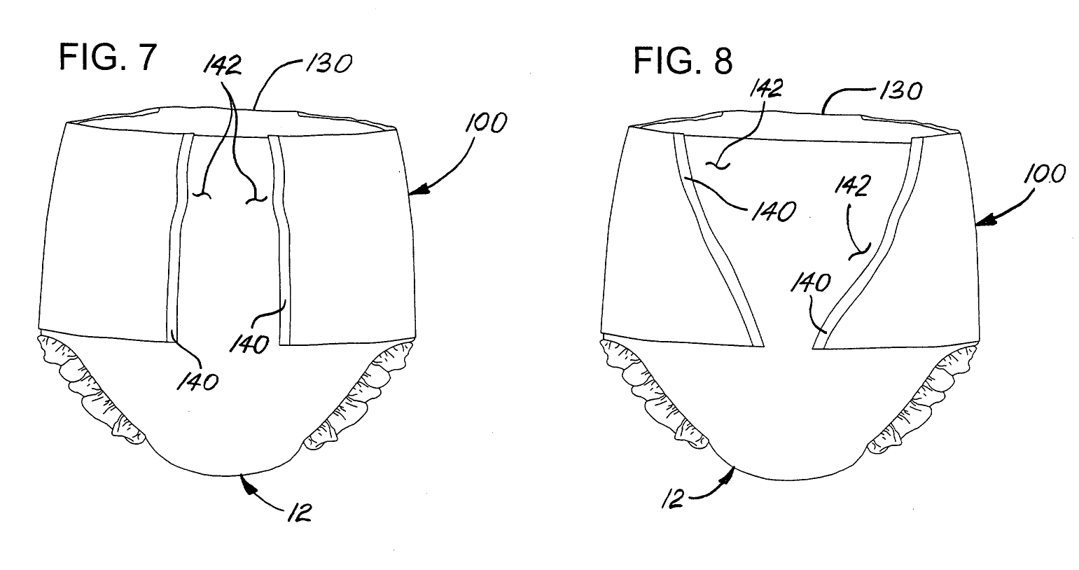

The fastening system 104 may have any of various configurations. In the embodiment shown in FIGS. 1-9, the system comprises cooperating first fastening components 140 on the support panel 100 and second fastening components 142 on the outer cover 30 of the front waist region 14 of the chassis 12. In certain configurations, the fastening system 104 is adapted for refastenably securing the training pant 10 about the waist of the wearer. However, it is contemplated that non-refastenable systems could also be used without departing from the scope of this invention. In one embodiment, one surface of each of the first fastening components 140 comprises a plurality of engaging elements which project from that surface. The engaging elements of the first fastening components 140 are adapted to repeatedly engage and disengage engaging elements of the second fastening components 142.

The fastening components 140, 142 can comprise separate elements bonded to the support panel 100 and front waist region 14 of the chassis 12, or they may be integrally formed with the support panel 100 and/or waist region 14. Thus, unless otherwise specified, the term “fastening component” includes separate components which function as fasteners, and one or areas or regions of materials (e.g., areas of the front waist region 14) which function as fasteners. Moreover, a single material can define multiple fastening components to the extent that different regions of the material function as separate fasteners. In the embodiment of FIG. 1, the first fastening components 140 on the support panel 100 are separate elements which extend laterally out beyond the side edges of the end sections 122 of the support panel, and the second fastening components 142 comprise regions of the outer cover 30 of the front waist region 14 overlapped by the first fastening components 140 when the support panel is fastened in place. Alternately, the first fastening components 140 can be separate elements on the inside surface 134 of the support panel 100. In another embodiment, the first fastening components 140 may comprise integral regions of the support panel 100 and the second fastening components 142 may comprise separate fastening elements attached to the outer cover 30 of the front waist region 14. In still another embodiment, the first second and second fastening components 140, 142 may comprise integral regions of the support panel 100 and front waist region 14, respectively.

The fastening components 140, 142 can comprise any refastenable fasteners suitable for absorbent articles, such as adhesive fasteners, cohesive fasteners, mechanical fasteners, or the like. In particular embodiments the fastening components comprise mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

The refastenable fastening system 104 allows for easy inspection of the interior space 54 of the pant 10. When necessary, the fastening system 104 also allows the pant 10 to be removed quickly and easily. This is particularly beneficial when the pant contains messy excrement. For training pant 10, the caregiver can completely remove the pant and replace it with a new one without having to remove the child's shoes and clothing.

In the illustrated embodiment, the first fastening components 140 comprise hook fasteners and the second fastening components 142 comprise complementary loop fasteners. Alternatively, the first fastening components 140 comprise loop fasteners and the second fastening components 142 comprise complementary hook fasteners; or the fastening components 140, 142 can comprise interlocking similar surface fasteners, adhesive or cohesive fastening elements such as an adhesive fastener and an adhesive-receptive landing zone or material; or the like. Although the training pant 10 illustrated in FIG. 1 shows the support panel 100 overlapping the front waist region 14 upon connection thereto, which is convenient, the training pant 10 can also be configured with a front support panel which can be folded down and stretched to overlap the back waist region 16. Alternatively, the pant 10 may be configured to have front and support panels both of which can be folded down and stretched around the hips and secured to one another in overlapping positions. One skilled in the art will recognize that the shape, density and polymer composition of the hooks and loops may be selected to obtain the desired level of engagement between the fastening components 140, 142. A more aggressive hook material may comprise a material with a greater average hook height, a greater percentage of directionally-aligned hooks, or a more aggressive hook shape.

Loop fasteners typically comprise a fabric or material having a base or backing structure and a plurality of loop members extending up from at least one surface of the backing structure. The loop material can be formed of any suitable material, such as acrylic, polyamide, polyethylene, polypropylene or polyester, and can be formed by methods such as warp knitting, stitch bonding or needle punching. Loop materials can also comprise any fibrous structure capable of entangling or catching hook materials, such as carded, spunbonded or other nonwoven webs or composites, including elastomeric and nonelastomeric composites. Suitable loop materials are available from Guilford Mills, Inc., Greensboro, N.C., U.S.A. under the trade designation No. 36549. Another suitable loop material can comprise a pattern un-bonded web as disclosed in U.S. Pat. No. 5,858,515 issued Jan. 12, 1999 to Stokes et al., the disclosure of which is incorporated herein by reference for all purposes not inconsistent with the present invention.

Hook fasteners typically comprise a fabric or material having a base or backing structure and a plurality of hook members extending upwardly from at least one surface of the backing structure. In contrast to the loop fasteners which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded from nylon, polypropylene, polyethylene or another suitable material. Suitable single-sided hook materials for the fastening components are available from commercial vendors such as Velcro Industries B.V., Amsterdam, Netherlands or affiliates thereof, and are identified as Velcro HTH-829 with a uni-directional hook pattern and having a thickness of about 0.9 millimeters (35 mils) and HTH-851 with a uni-directional hook pattern and having a thickness of about 0.5 millimeters (20 mils); and Minnesota Mining & Manufacturing Co., St. Paul, Minn. U.S.A., including specific materials identified as CS-600.

With particular reference to FIGS. 1-8, the first fastening components 140 are illustrated as being disposed along opposite side edges of the support panel 100, and the second fastening components 142 are disposed on the outer surface 30 of the front waist region 14 of the chassis 12. The second fastening components 142 can be sized to receive the first fastening components 140 when the support panel 100 is stretched to overlap the front waist region 14. Where the second fastening components 142 comprise loop fasteners disposed on the front waist region 14 and the first fastening components 140 comprise hook fasteners disposed on the support panel 100, the second fastening components 142 can be sized larger than the first fastening components to ensure coverage of the rigid, outwardly-directed hooks.

The fastening components 140, 142 can be adhered to the respective support panel 100 and waist region 14 by any means known to those skilled in the art such as adhesive bonds, ultrasonic bonds or thermal bonds. The fastening components 140, 142 may comprise separate fastening elements or distinct regions of an integral material. For example, the training pant 10 can include an integral second fastening material disposed in the front waist region 14 for refastenably connecting to the first fastening components 140 at two or more different regions, which define the second fastening components 142 (FIG. 1). In a particular embodiment, the fastening components 140, 142 can comprise integral portions of the support panel 100 and front waist region 14. For instance, the front waist region 14 can function as the second fastening components 142 in that it can comprise a material which is releasably engageable with fastening components 142 disposed on the support panel 100.

The fastening components 140, 142 of the illustrated embodiments are rectangular, although they may alternatively be square, round, oval, curved or otherwise non-rectangularly shaped. In particular embodiments, each of the fastening components 140, 142 has a length aligned generally parallel to the Y direction and a width aligned generally parallel to the X direction. For a child of about 9 to about 15 kilograms (20-30 pounds), for example, the length of the fastening components 140, 142 is desirably from about 50 to about 130 mm, such as about 100 mm, and the width is desirably from about 5 to about 30 mm, such as about 10 mm. With particular embodiments, the fastening components 140, 142 can have a length-to-width ratio of about 2 or greater, such as about 2 to about 25, and more particularly about 5 or greater, such as about 5 to about 8. For other embodiments such as for adult products, it may be desirable for one or more of the fastening components to comprise a plurality of relatively smaller fastening elements. In that case, a fastening component or individual fastening elements may have an even smaller length-to-width ratio, for example, of about 2 or less, and even about 1 or less.

The support panel 100 can be secured in different supporting positions, depending on the shape and size of the wearer and/or the desirable tension to be applied to the wearer. For example, by stretching the support panel 100 to a greater or lesser degree, the amount of force exerted by the panel on the chassis 12 can be varied as desired. Further, the tension exerted by the support panel 100 can be varied over the height of the support panel (i.e., in the Y direction) by adjusting the manner in which the fastener components 140, 142 are connected. For example, in FIG. 1, the support panel 100 is secured in a supporting position in which the fastening components 140 are generally parallel to the longitudinal direction Y. In FIG. 8, the fastening components 140 are angled so that the lower portion of the support panel 100 is stretched to a greater degree than the upper portion of the panel, which may be effective to tighten the pant around the lower hip of a wearer; and in FIG. 9, the fastening components 140 are angled so that the upper portion of the support panel 100 is stretched to a greater degree than the lower portion of the panel, which may be effective to tighten the pant around the waist of a wearer.

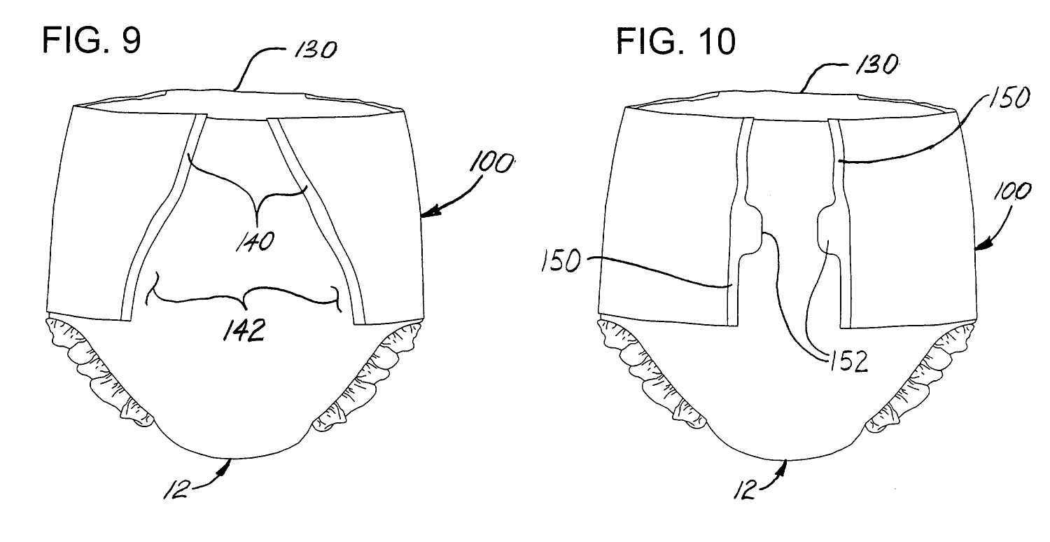

Other types of fastening arrangements are possible. For instance, FIG. 10 shows a fastener system comprising a pair of elongate substantially rigid fastening components 150 attached along opposite ends of the support panel 100, and thumb tabs 152 on the components 150 for pulling the ends of the support panel to the desired fastening positions. The fastening components 150 are formed with fastening elements (e.g., hooks) which interact with cooperating fastening elements (e.g., loops) on the front waist region 14 of the absorbent chassis to secure the support panel in position. The rigidity of the fastening components 150 (which may be referred to as “beams”) functions to distribute the tensioning forces exerted on and by the support panel 100 substantially evenly in the Y direction along the entire height of the support panel. FIG. 11 shows two fastening components 164 spaced along each side of the support panel 100. FIG. 12 shows the fastening system of FIG. 11 but with the lower fastening components 164 arranged to ease the tension across the lower hip. FIG. 13 shows a fastening system comprising three fastening components 168 spaced along each side of the support panel 100. FIG. 14 shows the fastening system of FIG. 13 but with the fastening components 168 arranged to tighten the pant at the waist and lower hip. Other arrangements are possible.

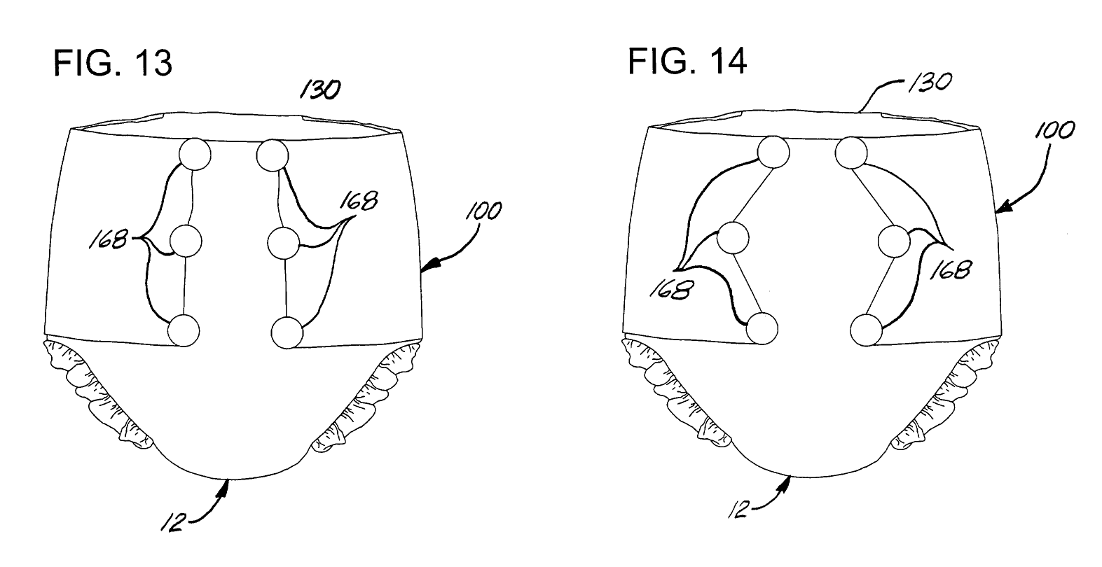

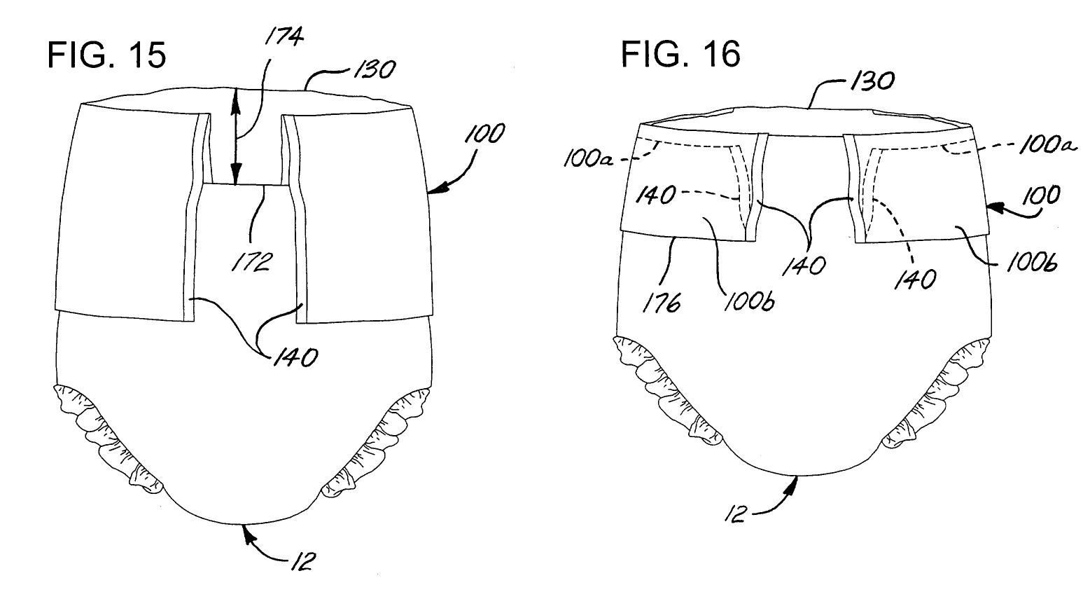

FIG. 15 illustrates a configuration where the support panel 100 is folded down along a fold line 130 spaced above the upper back waist edge 172 of the chassis a distance 174. The advantage of this design is that the portions of the support panel above the waist edge 172 form, in effect, a double-thickness elastic waist band which functions to provided an increased tension for assisting in holding the pant in place. This increased holding power can be especially advantageous where the body of the wearer has a shape (e.g., a pear shape or a large waist) tending to cause the pant to slip down on the body. FIG. 16 illustrates a configuration where a lower portion 100a of the folded-down support panel 100 is folded up on the underside of an upper portion 100b of the panel on a fold line 176, forming a support panel of double thickness to provide greater pressure against the wearer in the area of the double thickness. In this configuration the lower folded-up portion 100a of the support panel 100 is held in place only by the pressure exerted by the upper folded-down portion 100b of the panel. This configuration may be desirable for providing higher tensioning in certain areas, as where the wearer has a larger waist and narrower hips and more pressure is required at the waist to keep the pants from slipping down. FIG. 17 shows a similar folded-under configuration except using the double fastening components 164 of FIG. 11. In this embodiment, the lower two fastening components 164 are bent back on the underside of the lower portion 100a of the support panel 100 and the lower portion 100a is folded up under the upper portion 100b, thus rendering the two lower fastening components 164 non-functional in the sense that they do not fasten to the fastening components on the waist region 14 of the absorbent chassis. Alternatively, the two lower fastening components 164 could be on the opposite face of the support panel 100, thus rendering them functional (i.e., attachable to the fastening components on the waist region 14) when the lower portion 100a of the panel is folded up under the upper portion 100b.

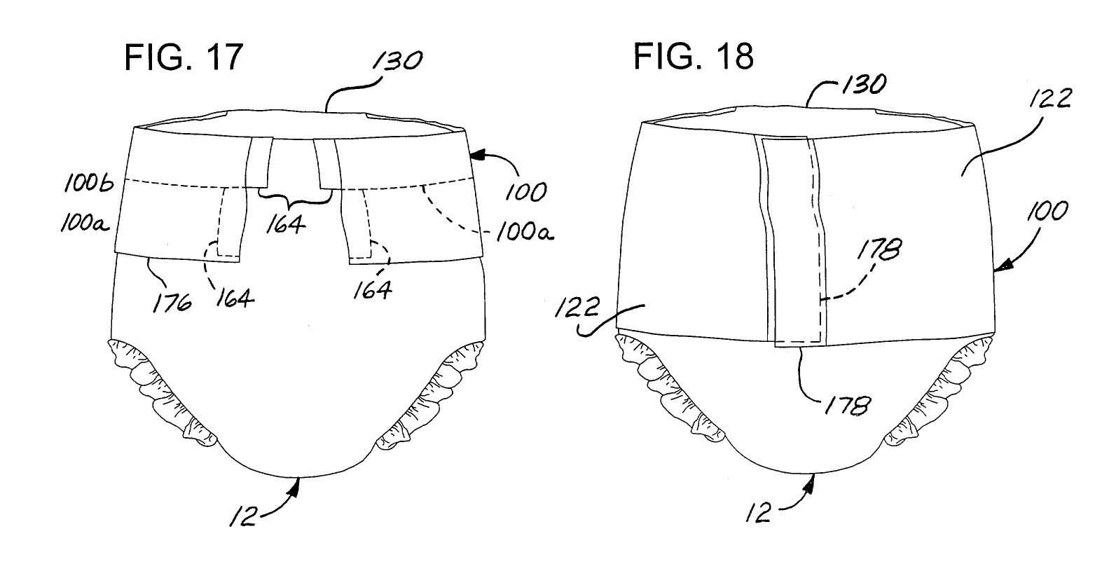

FIG. 18 shows a configuration where opposite end sections 122 of the support panel 100 overlap and include cooperating fastening components 178 which fasten to one another to maintain the panel in its supporting position.

The support panel 100 of this invention provides a number of advantages. It can be used with a full size chassis 12 to ensure that the garment has full absorbent functionality with no reduction in absorbent area. When the panel 100 is secured in its supporting position, it maintains pressure against the chassis 12 sustantially around the full circumference of the chassis to keep the leg openings 58 closed and the chassis tight against the body of the wearer without bunching of the chassis. As a result, the garment is more form fitting for enhanced containment and appearance. Further, since the support panel 100 functions independent of the chassis 12, the panel does not interfere with the movement of the wearer. Rather, it stretches to accommodate such movement while maintaining the chassis 12 close to the body of the wearer. As described above, the support panel 100 can be stretched and secured in a variety of supporting positions, depending on the shape, size and preferences of the wearer and/or caregiver.

Another advantage of the support panel 100 is that it can be made separately from the chassis 12. Consequently, the construction of the panel 100, the elastic characteristics of the panel, and the fastening components 140 on the panel can be tailored to meet specified performance criteria during manufacture and prior to attachment to the chassis 12. Further, the support panel 100 of this invention simplifies the time required to modify equipment to change over from one size pant to another. A change-over from one size training pant to another can require substantial equipment modification, since new side panels of different size must be added to the same chassis. No such addition is required with a training pant of the present invention. A supply of new support panels 100 is simply added to the line, typically as a web of connected panels fed from a roll. The same chassis 12 is used for the new size. This provides a significant advantage since different size garments may require different stretch capabilities (but not necessarily different absorption capabilities). Gradient tensioning or stretch differentials can be built into the panel and/or accompanying fastening components to accommodate different needs across the waist, hip and leg areas. Also, elastic patterning at the waist area can be manipulated to minimize attachment interactions and optimize waist functionality across both hips and back. For additional detail regarding exemplary targeted elastic arrangements, reference may be made to U.S. patent application Ser. No. 09/855,189 by Carr et al. titled “Targeted Elastic Laminate Having Zones of Different Basis Weights”, filed May 14, 2001 and published as U.S. Publication Number 2002-0002021, and U.S. patent application Ser. No. 09/855,188 by Salter et al. titled “Targeted Elastic Laminate Having Zones of Different Polymer Materials”, filed May 14, 2001 and published as U.S. Publication Number 2002-0009940, both of which are incorporated herein by reference for all purposes not inconsistent herewith.

The various elements of the training pant 10 can be connected together by any means known to those skilled in the art such as, for example, adhesive, thermal and/or ultrasonic bonds. Desirably, most of the elements are connected using ultrasonic bonding for improved manufacturing efficiency and reduced raw material costs.

The training pant 10 can be made using various processes. In one embodiment, the chassis 12 is made in a cross-direction process as a separate entity and having full absorbent functionality with only the leg and/or flap elastics in place. (There is no need for conventional waist elastics.) The support panel 100 is made off line and delivered to the process line in roll form. During unwind, the fastening components 140 are attached to the support panel 100, which is then attached to a waist region (e.g., back waist region 14) of the chassis 12. The support panel can be applied in either a relaxed or tensioned state, depending on the elastics patterning near the area of attachment to the waist region and the method of attachment. For example, if the center section 120 of the support panel 100 is inelastic, or if sections (e.g., end sections 122) of the support panel are attached to the waist region 14 by frangible connections, no tensioning of the panel at the time of attachment is required. An alternate method of assembly can be used in a machine direction process. A 90-degree turn roll can be used to turn and position the support panel 100 along the waist region 14 of the chassis 12. Suitable mechanisms useful for this application are described in detail in U.S. Pat. No. 4,608,115, granted Aug. 26, 1986 to Schroth et al.; U.S. Pat. No. 5,104,116, granted Apr. 14, 1992 to Pohjola; U.S. Pat. No. 5,224,405, granted Jul. 6, 1993 1992 to Pohjola; U.S. Pat. No. 5,716,478, granted Feb. 10, 1998 to Boothe et al.; U.S. Pat. No. 5,759,340, granted Jun. 2, 1998 to Boothe et al.; U.S. Pat. No. 6,139,004, granted Oct. 31, 2000 to Couillard et al.; and U.S. Pat. No. 6,227,541, granted May 8, 2001 to Couillard et al.; all of which are incorporated herein by reference for all purposes not inconsistent herewith.

While the above description is primarily directed to training pants, it will be understood that the teachings of this invention are also applicable to diapers, adult care products and the like.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

1. A disposable absorbent garment comprising
an absorbent chassis having a front waist region, a back waist region and a crotch region extending longitudinally therebetween,
a support panel extending longitudinally from one of the waist regions of the absorbent chassis and adapted to be positioned in a folded-down condition in which the support panel overlaps said one waist region on the outside of the absorbent chassis, said support panel being elastically stretchable in a direction generally transverse to a longitudinal axis of the absorbent chassis, and
a fastening system for securing the support panel in a chassis supporting position in which the support panel is in said folded-down condition and elastically stretched girthwise around opposite hips of a person wearing the garment to support said absorbent chassis on the person.

2. A garment as set forth in claim 1 wherein said support panel extends longitudinally from said back waist region.

3. A garment as set forth in claim 2 wherein opposite end sections of said support panel overlap the front waist region of the absorbent chassis when the support panel is secured in said chassis supporting position.

4. A garment as set forth in claim 3 wherein said fastening system comprises cooperating fastening components on the support panel and on said front waist region.

5. A garment as set forth in claim 4 wherein said support panel in said folded-down condition has an inner surface facing toward said back waist region of the absorbent chassis and an outer surface facing away from the back waist region, said fastening components comprising a first set of hook fastening components on said support panel and a second set of loop fastening components on an outer surface of said front waist region.

6. A garment as set forth in claim 1 wherein said support panel comprises elastic elements laminated between inner and outer facing layers.

7. A garment as set forth in claim 6 wherein said inner and outer facing layers are comprised of a non-woven material.

8. A garment as set forth in claim 1 wherein said support panel in said chassis supporting postition is adapted to encircle at least two-thirds the circumference of the waist of the wearer.

9. A garment as set forth in claim 1 wherein said garment is a training pant, and wherein said back waist region has back ear portions extending laterally outward at opposite sides of the back waist region, said support panel extending from the back waist region and having a relaxed width not less than the overall width of the back waist region and the back ear portions.

10. A training pant as set forth in claim 9 wherein opposite ends of said support panel overlap said front waist region of the absorbent chassis when the support panel is secured in said chassis supporting position.

11. A training pant as set forth in claim 10 wherein said fastening system comprises cooperating fastening components on the support panel and on the front waist region.

12. A training pant as set forth in claim 11 wherein said support panel in said folded-down condition has an inner surface facing toward the back waist region of the absorbent chassis and an outer surface facing away from back waist region, said fastening components of the fastening system comprising a first set of hook fastening components on said support panel and a second set of loop fastening components on an outer surface of said front waist region.

13. A garment as set forth in claim 1 wherein said garment is a diaper.

14. A garment as set forth in claim 1 wherein said fastening system is adjustable to secure the support panel in a position in which the tension in the support panel varies over the height of the support panel.

15. A garment as set forth in claim 1 wherein a lower portion of the support panel is adapted to be folded up under an upper portion of the support panel when the support panel is in its folded-down condition.

16. A garment as set forth in claim 1 wherein each waist region has a longitudinal end, and wherein the longitudinal end of said one waist region is narrower than the longitudinal end of the other waist region when the garment is in a relaxed condition.

17. A garment as set forth in claim 1 wherein said garment is an adult incontinence garment.

18. A disposable absorbent garment comprising
an absorbent chassis having a front waist region, a back waist region and a crotch region extending longitudinally therebetween,
said chassis having lateral sides which define, at least partially, leg openings when the garment is worn,
leg elastic members along the lateral sides of the chassis adjacent the leg openings,
a support panel extending longitudinally from one of the waist regions of the absorbent chassis and adapted to be positioned in a folded-down condition in which the support panel overlaps said one waist region on the outside of the absorbent chassis, said support panel being elastically stretchable in a direction generally transverse to a longitudinal axis of the absorbent chassis, and
a fastening system for securing the support panel in a chassis supporting position in which the support panel is in said folded-down condition and elastically stretched girthwise around opposite hips of a person wearing the garment to support said absorbent chassis on the person, said support panel and said leg elastic members combining to substantially completely surround said leg openings.

19. A disposable training pant comprising
an absorbent chassis having a front waist region, a back waist region and a crotch region extending longitudinally therebetween,
a back support panel extending longitudinally from the back waist region of the absorbent chassis and adapted to be positioned in a folded-down condition in which the support panel overlaps the back waist region on the outside of the absorbent chassis, said support panel being elastically stretchable in a direction generally transverse to a longitudinal axis of the absorbent chassis, and
a fastening system for securing the back support panel in a chassis supporting position in which the back support panel is in said folded-down condition and elastically stretched girthwise around opposite hips of a person wearing the garment to support said absorbent chassis on the person.

20. A training pant as set forth in claim 19 wherein said back waist region has a longitudinal end extending transverse to said longitudinal axis, said support panel having a center section generally coextensive with said longitudinal end and two end sections extending from opposite sides of the center section.

21. A training pant as set forth in claim 20 wherein said longitudinal end of the back waist region has gathers formed therealong when the support panel is in a relaxed condition.
